(19) United States
(12) Patent Application Publication
(10) Pub. No.: US 2020/0121585 A1
(43) Pub. Date: Apr. 23, 2020

(54) Unit for the manufacture of an extract of interest of at least one plant, microorganism and/or fungus

(71) Applicant: CAPSUM, Marseille (FR)

(72) Inventors: Sébastien BARDON, Paris (FR); Mathieu GOUTAYER, Saint Malo (FR); Benjamin AUGAIS, Allauch (FR)

(21) Appl. No.: 16/661,739

(22) Filed: Oct. 23, 2019

(30) Foreign Application Priority Data
Oct. 23, 2018 (FR) 18 71277

(51) Int. Cl. A61K 8/9789; A61K 8/99; A61K 8/9728

(52) U.S. Cl. CPC A61K 8/9789; A61K 8/9728; A61K 8/99

(57) ABSTRACT

A unit for producing at least one extract of interest from at least one plant, at least one microorganism and/or at least one fungus, comprising at least two sections: a first section dedicated to the cultivation, under controlled conditions, of at least one plant, at least one microorganism and/or at least one fungus; and a second section dedicated to the production of the extract of interest derived from the plant and/or the microorganism and/or the fungus.

The present invention relates to a novel ecological and sustainable mode for producing an extract of interest derived from at least one plant and/or at least one microorganism and/or at least one fungus, even for manufacturing a composition particularly a cosmetic composition comprising said extract of interest.

Cosmetics, like other mass-produced industrial products are essentially based on chemistry and complex manufacturing logistics generally removed and even hardly compatible with notions of ecology and sustainable development.

However, and irrespective of sector, an ever-growing consumer demand is observed for «sustainable» products, derived in particular from organic farming without pesticides and/or with short supply chains.

In cosmetics, efforts have been made in this direction. As an illustration, an increasing number of active agents are derived from the extraction of natural products, and in addition using so-called «eco-extraction» methods paying heed to the environment. Also, labels such as COSMEBIO value cosmetic products comprising ingredients derived from organic cropping. Mention can also be made of the labels NATURE ET PROGRES, ECOCERT, NA TRUE or BDIH, which focus on «organic» cosmetics.

It nevertheless remains that obstacles and/or restrictions, particularly industrial, which become more heightened for complex compositions such as emulsions, are likely to have a non-negligible impact on the technical feasibility and/or economic soundness of an ecological and sustainable project, in particular when applied to the cosmetic field.

It is one objective of the invention to provide a tool for producing an extract of interest from at least one plant and/or at least one microorganism and/or at least one fungus, even a composition particularly a cosmetic composition comprising said extract of interest, which does not have the above-mentioned drawbacks.

In particular, it is one objective of the invention to provide an ecological and sustainable tool for producing an extract of interest from at least one plant and/or at least one microorganism and/or at least one fungus, even a composition, particularly a cosmetic composition, comprising said extract of interest.

The invention concerns a unit for producing at least one extract of interest from at least one plant and/or at least one microorganism and/or at least one fungus, comprising at least two sections:
a first section dedicated to the cultivation, under controlled conditions, of at least one plant and/or at least one microorganism and/or at least one fungus; and
a second section dedicated to the production of the extract of interest derived from said plant and/or said microorganism and/or said fungus.

In a first embodiment, the unit also comprises a third section differing from the above-mentioned first and second sections and dedicated to the manufacture of a composition particularly a cosmetic composition comprising at least the extract of interest and a physiologically acceptable medium.

A unit of the invention in this first embodiment therefore allows extemporaneous preparation of a composition particularly a cosmetic composition comprising at least the extract of interest and a physiologically acceptable medium. Said unit is therefore a unit for the production of (i) at least one extract of interest from at least one plant and/or at least one microorganism and/or at least one fungus and of (ii) a composition, particularly a cosmetic composition, comprising at least the extract of interest and a physiologically acceptable medium.

In a second embodiment, the unit comprising the third section also comprises a fourth section differing from the above-mentioned first section, second section and third section, and dedicated to the packaging of said composition, particularly a cosmetic composition, in a container.

In a third embodiment, the unit also comprises a fifth section differing from the above-mentioned first and second sections and also third and fourth sections if any, and dedicated to the marketing of the extract of interest or of the composition manufactured in the third section or packaged in the fourth section.

The invention also concerns the use of a unit of the invention to produce an extract of interest, even a composition, particularly a cosmetic composition, comprising at least said extract of interest and a physiologically acceptable medium.

The invention further concerns the use of at least one extract of interest obtained by means of a unit of the invention for the preparation of a composition, particularly a cosmetic composition.

The invention also concerns a method for producing at least one extract of interest from at least one plant and/or at least one microorganism and/or at least one fungus, said method comprising at least the steps of:

(a) providing at least one unit of the invention;

(b) cultivating at least one plant and/or at least one microorganism and/or at least one fungus under controlled conditions;

(c) extracting the extract of interest from the plant and/or microorganism and/or fungus harvested at step (b);

(d) optionally, manufacturing a composition particularly a cosmetic composition comprising at least the extract of interest and a physiologically acceptable medium; and

(e) optionally packaging said composition in a container;

at least steps (b) to (c) being conducted within the unit.

A method of the invention comprises a step to harvest the plant and/or microorganism and/or fungus between steps (b) and (c).

Preferably, steps (b) to (d), even (b) to (e), are conducted within a unit of the invention.

If necessary, a method of the invention between steps (b) and (c) may also comprise at least one intermediate step (f) dedicated to storage and/or preparation for the extraction step (c). This intermediate step (f) can be a freezing, dry-freezing, drying, grinding and/or centrifugation step of the plant and/or microorganism and/or fungus.

For a plant, grinding particularly comprises the «grinding filtration» technique whereby the «juice» of a plant is separated from the fibres and other solid elements of greater size than the pores of the grinding filter, extraction then being performed from the «juice».

Preferably, a method of the invention does not comprise a step, and in particular an intermediate step (f), involving freezing and/or dry-freezing. The unit and a method of the invention are adapted for extraction from a fresh plant and/or microorganism and/or fungus.

Persons skilled in the art are able to adjust/choose step (f), in particular having regard to the plant, microorganism and/or fungus under consideration.

As follows from the description, the present invention is advantageous at least for the following reasons:
the cultivation of plants and/or microorganisms and/or fungi is conducted under controlled conditions, allowing the risk of diseases and/or contamination to be reduced and even prevented;
the cultivation of plants and/or fungi under controlled conditions therefore allows limited recourse to pesticides, and even eliminates any need thereof. In addition, the unit provides against any contamination by pesticides derived from neighbouring crops;
growth of the plants and/or fungi is better controlled and can be conducted all year round with exceptional production yields;
the unit is not subject to sudden changes in temperature, rain, hail, frost or predatory insects, thereby significantly reducing risks of the plants and/or fungi being destroyed with consequent loss of harvest;
the unit can be remotely computer-controlled;
the unit ensures extraction on an ultra-fresh plant, microorganism and/or fungus, hence with high content of active substance of interest, and additioinally in non-degraded form.
the unit enables a cultivation mode of plants, microorganisms and/or fungi that is practically self-sufficient in elements required for their survival and growth, in particular regarding water, light, oxygen and/or temperature;
the unit allows a reduction in the needs of elements required for growth of the plants and/fungi, in particular a reduced water supply that can feed the plants and/or fungi in a closed circuit;
the unit allows the steps of cultivation, extraction and even optionally production and marketing of the end product within a much-restricted geographical area, possibly in an urban environment and therefore close to final consumers allowing the provision of a short supply chain;
the unit allows the recycling of some organic waste and excreta derived in particular from the cultivation and extraction steps, to contribute towards all or part of the growth of future generations of plants and/or microorganisms and/or fungi; and
the unit allows limited use of transport and storage chains that are costly in vehicles, machines, fuel and packaging, thereby bringing a reduction in CO2 emissions and energy and fossil carbon needs, hence reduced costs and environmental impact.

Unless otherwise stated, the steps described in the present description are conducted at ambient temperature (e.g. T=25° C.±2° C.) and atmospheric pressure (760 mm of Hg, i.e. 1,013.10⁵ Pa or 10¹³ mbar).

Also, in the meaning of the present invention, an extract of interest is an active agent. In the present description, by «active agent 141 it is meant a compound having a beneficial physiological effect on the element on which it acts. For example, it is intended to protect, maintain in good condition, treat, cure, perfume, flavour or colour. The extract of interest is advantageously a cosmetic, dermo-pharmaceutical, pharmaceutical or food agent.

Therefore, in the present invention, the term «extract of interest» can indifferently designate the term “active agent”.

Preferably, a composition of the invention is free of surfactant.

A unit of the invention is dedicated to the production of at least one extract of interest from at least one plant and/or at least one microorganism and/or at least one fungus, even of a composition, particularly a cosmetic composition, comprising at least said extract of interest and a physiologically acceptable medium. For this purpose, a unit of the invention comprises at least the above-mentioned first and second sections, even optionally the third section, fourth section and fifth section.

In the meaning of the present invention, a unit designates a geographical area of restricted perimeter, for example having a surface area of less than 10 000 m², preferably less than 5 000 m², in particular less than 2 500 m², better still less than 1 000 m², preferably less than 500 m², in particular less than 100 m² and more particularly less than 50 m².

Preferably, a unit is represented by at least one building (or enclosure). A unit of the invention can also be formed of at least two inter-connected buildings.

In particular, the unit is formed of at least one shipping container reconditioned to receive at least the first section and the second section, even optionally the above-mentioned third section, fourth section and fifth section.

In particular, the unit is a retail outlet dedicated to the marketing of the extract of interest, even of the composition particularly a cosmetic composition comprising at least said extract of interest and a physiologically acceptable medium.

Preferably, the unit is adapted to ensure total control over the cultivation conditions, in particular to ensure controlled regulation of water, hygrometry, temperature, air quality and provision of light.

Therefore, preferably, a unit of the invention is an impervious (or sterile) enclosure at least in the first section. In other words, in a unit of the invention at least the first section is adapted for the cultivation of plants and/or microorganisms and/or fungi under controlled conditions.

By «controlled conditions» in the present invention it is meant to designate an enclosure in which at least the hydric and hydrometric parameters, luminosity, temperature and air quality are mastered and controlled, possibly computer-controlled.

Regarding the hydric parameter, the water used to cultivate the plant, microorganism and/or fungus can be selected from among any suitable water source, and can be selected in particular from among mains water, rainwater, aquifer ground water. Optionally, or if necessary, the water can be treated prior to use, in particular to remove any trace of contamination especially of pesticides.

In particular, the water is depolluted using ultraviolet radiation, filters preferably having pores with diameter of 0.2 micron, self-purifying microorganisms such as Daphnia (also known as «water fleas»), and mixtures thereof.

Regarding luminosity, it is possible to use sunlight and/or artificial light particularly using LED technology. If sunlight is used, the unit can be adapted notably through the construction of a transparent roof.

Regarding the atmospheric parameter, at least the first section of the unit can be adapted for the injection of quality air, of controlled temperature and/or humidity.

Regarding the temperature parameter, it is possible to have recourse to a heating or air-conditioning system, that is possibly reversible.

Additionally, the operation of the unit is advantageously adapted to be self-sufficient i.e. it produces the energy source(s) required for such operation.

In particular, the energy used to ensure the functioning of a unit of the invention is clean energy (also called green energy) i.e. a primary energy source which produces a small amount of pollutants when converted to final power and used as such.

Preferably, the energy used to ensure the functioning of a unit of the invention is selected from among solar energy including photovoltaic and thermal energy, wind energy, tidal energy, wave energy, hydroelectric energy, geothermal power, spatial solar energy, nuclear energy and biomass, preferably solar energy.

It is within the reach of skilled persons to make the necessary adjustments to the unit, at least in the first section, to fulfil the criterion of «controlled conditions» as required by the invention, and to ensure cultivation of the plant, microorganism and/or fungus, and if needed to enable the unit to produce its own source(s) of energy.

### First Section

Cultivation of the plant and/or microorganism and/or fungus can be carried out using any known method. Preferably, the cultivation method is adapted to the configuration and restricted perimeter of a unit of the invention, and to the plant and/or microorganism and/or fungus it is desired to cultivate (grow).

In this respect, for plants and/or fungi, one particularly well-suited cultivation method can be of «urban farming» type (also called URBAN VERTICAL FARMING SYSTEM) based on the principle of hydroponics even aquaponics.

For example, in the first section of a unit of the invention, the cultivation of the plant and/or fungus is hydroponic or aquaponic cultivation.

As an illustration, an appropriate farming system is a hydroponic system such as described for example in EP0010836, WO1996032835, WO2016072042, US2017049064.

Another suitable farming system is an aquaponic system such as described for example in WO2015160966 or WO2008006172.

For plant cells and/or microorganisms, one particularly well-suited culture method can be fermentation or encapsulation.

The culture of the microorganism(s) under consideration can be conducted aerobically in a suitable (or adequate) culture medium e.g. under the conditions described in documents U.S. Pat. No. 4,464,362 and EP0043128. When the stationary development phase is reached, the culture medium can be inactivated by pasteurisation e.g. at a temperature of 60 to 65° C. for 30 minutes. The microorganisms are collected using a conventional separation technique e.g. membrane filtration, centrifugation and resuspended in a solution of sterile physiological NaCl.

“Fermentation” is generally conducted in fermenters with a suitable culture medium, adapted to the microorganism being cultivated, containing at least one simple carbon source and co-substrates if needed.

A “suitable culture medium” designates a medium (e.g. a sterile liquid medium) comprising nutrients that are essential or useful for the maintaining and/or growth of microorganisms, such as carbon sources or carbon-containing substrates, sources of nitrogen e.g. peptone, yeast extracts, meat extracts, malt extracts, urea, ammonium sulfate, ammonium chloride, ammonium nitrate and ammonium phosphate; sources of phosphorus e.g. monopotassium phosphate or dipotassium phosphate; trace elements (e.g. metal salts) for example magnesium salts, cobalt salts and/or manganese salts; and growth factors such as amino acids, vitamins, growth promoters etc. The term “carbon source” or “carbon-containing substrate” or “carbon source” designates any carbon source able to be used by skilled persons to promote normal growth of a microorganism, including hexoses (such as in the form of glucose, galactose or lactose), pentoses, monosaccharides, oligosaccharides, disaccharides (such as sucrose, cellobiose or maltose), molasses, starch or derivatives, cellulose, hemicelluloses and mixtures thereof.

A suitable encapsulation system can be the one described for example in WO2016062836. The encapsulation of plant cells and/or microorganisms and culture in capsules has the following advantages:
the encapsulated plant cells and/or microorganisms are protected against mechanical stresses such as shear, thereby reducing cell death throughout culture;
the encapsulated plant cells and/or microorganisms are partly protected against the outside medium since the membrane of the capsule has selective permeability (in particular bacteria cannot enter into the capsule), which advantageously allows the preventing of any contamination and hence reduces cell death;
the size of the capsules (several hundred micrometres) and their mechanical properties make them easier to handle than algae, in particular for changes of external culture medium or when harvesting; and
post-treatment after growth can be performed to make the capsules impervious thereby isolating the content thereof to facilitate storage until use.

A unit of the invention comprises at least one first section. A unit may also comprise a plurality of first sections, in particular at least two first sections.

In one particular embodiment, at least two different living organisms from among a plant, microorganism and/or fungus are simultaneously cultivated in a unit of the invention. For illustration, at least two different plant species, at least two different microorganism species, or at least one plant species and at least one microorganism or fungus species are simultaneously cultivated in a unit of the invention.

In a first variant, the unit then comprises at least two first sections, each first section comprising a plant species, a microorganism species or fungus species differing from the plant, microorganism or fungus species contained in the second first section, or one of the first sections comprises a plant and the other first section comprises a microorganism or fungus.

In a second variant, the unit comprises a single first section in which at least two plant species, at least two microorganism species, at least two fungus species, or at least one plant species and at least one microorganism or fungus species are cultivated together.

A unit of the invention is therefore adapted for symbiotic association, also called mycorrhization, e.g. between at least two plant species or between at least one plant species and at least one microorganism or fungus species.

This particular embodiment is advantageous since, within one same unit, it is possible to produce at least two different extracts of interest, or one extract of interest produced solely in the presence of a symbiotic relationship between at least two different plant species or between at least one plant species and at least one microorganism or fungus species.

A unit of the invention is adapted for the cultivation of all types of plants, fungi, microorganisms, and mixtures thereof.

### Plants

A unit of the invention is adapted for the culture of any type of plant selected in particular from among mosses, ferns, gymnosperms and angiosperms; algae; and mixtures thereof.

A plant can be selected from among garlic, alfalfa (or lucerne), almond, amaranth, dill, arugula, oats, azukis, basil, Swiss chard (oriole or pink), beetroot, leaf beet, wheat, broccoli, kaniwa (baby quinoa), carrots, celery, chervil, chia, chicory, cabbage, scallions, chives, coriander, watercress, lady's smock, spelt, spinach, fennel, fenugreek, squash, sunflower, mung bean, kale (or curly kale), oriental wheat, lentils, red lentils, green lentils, flax, corn, sorghum, mizuna, mustard, turnip, onion, barley, parsley, leek, chick peas, peas, purslane, quinoa, radish, horseradish, rice, rocket, buckwheat, rye, sesame, soybean, tatsoi, trefoil, cannabis, and mixtures thereof.

### Microorganism

A unit of the invention is adapted for the culture of any type of microorganism, selected in particular from among bacteria, yeasts and mixtures thereof, preferably bacteria.

The microorganisms of the invention can be selected from among prokaryote microorganisms, eukaryote microorganisms and mixtures thereof.

In particular, a unit of the invention is adapted for the culture of microbiome bacteria, particularly the human microbiome and/or bacteria of intestinal flora, in particular human intestinal flora.

A microorganism of the invention can be selected from among the genii Saccharomyces, Yarrowia, Kluyveromyces, Torulaspora, Schizosaccharomyces, Debaromyces, Candida, Pichia, Aspergillus, Penicillium, Bacteroides, Fusobacterium, Melissococcus, Propionibacterium, Enterococcus, Lactococcus, Staphylococcus, Peptostrepococcus, Bacillus, Pediococcus, Vitreoscilla, Micrococcus, Leuconostoc, Weissella, Aerococcus, Oenococcus, Bifidobacterium, and Lactobacillus, and mixtures thereof.

In particular, a microorganism of the invention can be selected from among the species Yarrowia lipolitica, Kluyveromyces lactis, Saccharomyces cereviseae, Schizosaccharamyces pombe, Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus paracasei, Lactobacillus rhamnosus (Lactobacillus GG), Lactobacillus sake, Lactococcus lactis, Streptococcus thermophilus, Staphylococccus carnosus, Staphylococcus xylosus, Bifidobacterium longum, Bifidobacterium bifidum, Bifidobacterium breve, Bifidobacterium animalis, Bifidobacterium lactis, Bifidobacterium infantis, Bifidobacterium adolescentis, Bifidobacterium pseudocatenulatum, Vitreoscilla filiformis, and mixtures thereof.

### Fungi

A unit of the invention is adapted for the cultivation of any type of fungus, selected in particular from among the Fungi (also called Mycota, Mycetes), oomycetes, chytridiomycetes (also called Chytridiomycota or chytrids), mycetozoa and mixtures thereof, preferably Fungi.

A fungus can be selected from among the genii Agaricus, Lentinula, Pleurotus, Grifola, Hypsizygus, Auricularia, Flammulina, Volvariella, Agrocybe, Pholiota, Tricholoma, Tremella, and mixtures thereof.

In particular, a fungus can be selected from among the species Agaricus bisporus (Common mushroom), Lentinula edodes (forest mushroom or shiitake mushroom), Pleurotus ostreatus (pearl oyster mushroom and other neighbouring species), Pleurotus eryngii (king trumpet mushroom), Grifola frondosa (hen-of-the -woods), Hypsizygus tessulatus (buna-shimeji), Auricularia auricula-judge (wood ear), Flammulina velutipes (velvet shank mushroom), Volvariella volvacea (straw mushroom), Agrocybe aegerita (poplar mushroom), Pholiota nameko (Chinese Huazimo), Tricholoma matsutake (matsutake), Tremella fuciformis (snow fungus), and mixtures thereof.

The cultivation and harvesting (e.g. picking, lifting, gathering) of the plant, microorganism and/or fungus can be carried out using any technique known to skilled persons and within their general knowledge.

### Second Section

The second section of a unit of the invention is dedicated to the production of at least one extract of interest derived from at least one plant and/or at least one microorganism and/or at least one fungus in the previously described first section.

In a first embodiment, the first section and the second section are located in the same building (or enclosure).

In another embodiment, the first section and the second section are located in two separate areas, the second section being adjacent to the first section however.

A unit comprises at least one second section. In a variant of embodiment, a unit may comprise at least two second sections, each second section able to allow the production of at least one different extract of interest. The extracts of interest differ from one another with respect to the plant and/or microorganism and/or fungus from which they are derived and/or from the extraction method under consideration.

Therefore, the harvesting of a plant and/or microorganism and/or fungus, the optional transforming thereof in particular with a view to extraction, and the production of the extract of interest only need a short logistic chain, hence with savings in time and in human, material and energy resources.

This configuration of the unit of the invention advantageously allows extraction to be performed on a fresh plant, microorganism and/or fungus, making it possible to reduce and even prevent degradation of production yield and/or of the quality of the extract of interest. With regard to plants in particular, it is known that some active agents are difficult to store over time. This is particularly due to the setting-up of biochemical tolerance mechanisms against abiotic stresses e.g. hydric and/or oxidative which can lead to the deterioration or consumption of said active agents by the plant itself, or to the production of non-desirable molecules likely to interact negatively with the active agent of interest. Yet, for obvious reasons, the longer the time interval between the harvesting of a plant, microorganism and/or fungus and the extraction of an active agent contained therein, the more the production yield and/or quality of said active agent can deteriorate.

By «fresh plant», in the meaning of the present invention it is meant a plant which has been harvested (e.g. picked, plucked or lifted from the ground) less than 24 hours, preferably less than 12 hours, in particular less than 5 hours, better still less than 3 hours even less than 1 hour before the extraction step. The above definition of a fresh plant applies mutatis mutandis to a fresh microorganism and a fresh fungus.

Therefore, in the meaning of the present invention, the «extract of interest» can be termed an «extemporaneous extract of interest» insofar as it is produced within a very short time interval between the harvesting of the plant, microorganism and/or fungus containing the extract of interest and the extraction thereof.

An extemporaneous extract of interest is therefore obtained from at least one plant, from at least one microorganism and/or from at least one fungus, or from at least one part of said plant, of said microorganism and/or said fungus, the extraction step of said active agent being performed rapidly even immediately after harvesting.

Optionally, between cultivation of the plant and/or microorganism and/or fungus and the extraction of the extract of interest, the plant and/or microorganism and/or fungus can be subjected to at least one intermediate step dedicated to preparing said plant and/or said microorganism and/or said fungus for extraction.

For example, this intermediate step may entail freezing, freeze-drying, centrifugation, drying and/or grinding of the plant and/or microorganism and/or fungus. This intermediate step can be performed in a first section and/or second section, even in an additional section differing from the above-mentioned first section, second section, third section, fourth section and fifth section.

Preferably, a unit of the invention does not comprise a freezing, freeze-drying or drying step of the plant, microorganism and/or fungus prior to the extraction of the extract of interest.

This advantage follows from the configuration of the unit which allows easy, rapid succession of the steps between cultivation, harvesting and extraction.

### Extraction

The extract of interest can be obtained from any material derived from all or part of at least one plant, at least one microorganism and/or at least one fungus.

The extract of interest can be obtained from any plant material derived from a whole plant, or from any part of said plant e.g. the leaves, stems, flowers and flowering tops, seeds, buds, roots, undifferentiated cells or stem cells of plants, and mixtures thereof.

In one particular embodiment, extraction is carried out on a sprouted seed (also called germination), a microgreen or young shoot, generally with soil-free cultivation. Preferably, the extract of interest is therefore obtained from a sprouted seed, a microgreen or young shoot, preferably a microgreen.

A sprouted seed is a very early stage of growth when the plant has a root system, the cotyledon leaves being little or not developed. The average cultivation time for most sprouted seeds is less than 10 days, even less than 5 days, starting from seeding up until harvesting. Sprouted seeds have properties, in particular nutritional properties, that are higher than those of dry seeds: the levels of vitamins and enzymes can be strongly increased during the germination process.

A sprouted seed is therefore a plant maintaining its root system and having earlier maturity than a microgreen.

A microgreen is a vegetable green harvested just after shoots have appeared. A microgreen has a single central stem which is cut just above the soil line when harvesting (therefore without the roots). The cotyledon leaves are fully developed and it generally has a pair of very small, partly developed true leaves. The average cultivation time for most microgreens is 10 to 14 days starting from seeding up until harvesting.

A young shoot is at an early maturity stage but later than a sprouted seed and microgreen. The average cultivation time for most young shoots is longer than 10 days, even longer than 15 days starting from seeding up until harvesting.

In general, sprouted seeds, microgreens and young shoots contain considerably higher levels of vitamins and carotenoids than their homologous mature plants.

The very short cultivation time, very small size, and high concentration of active agent(s) mean that sprouted seeds, microgreens and young shoots, preferably microgreens, are types of plants that are fully adapted for the present invention.

The extract of interest can also be obtained from any material derived in full or in part from at least one microorganism, said extract of interest possibly being naturally excreted by the microorganism or derived from disruption of the microorganism using any method known to skilled persons.

In one variant of the invention, the microorganism used for the extraction step is used in living, semi-active, inactivated or dead form.

In the meaning of the invention, an “inactivated” microorganism is a microorganism that is no longer capable in a culture of either temporarily or definitively forming colonies.

In the meaning of the invention, a “dead” microorganism is a microorganism that is definitively no longer capable in a culture of forming colonies.

Dead or inactivated microorganisms can have intact or ruptured cell membranes. Therefore, the term “inactivated” also designates extracts and lysates of microorganisms i.e. comprising fractions and/or metabolites of microorganism(s). The obtaining of dead or inactivated microorganisms can be carried out with any method known to skilled persons.

A lysate in the meaning of the invention commonly designates a material obtained after disruption or dissolution of biological cells via a phenomenon known as cell lysis, causing release of the intracellular biological constituents naturally contained in the cells of the microorganism under consideration as well as fragments of cell membrane components. In the meaning of the present invention, the term “lysate” is used indifferently to designate the entirety of the lysate obtained by inactivation of the microorganism under consideration or only a fraction thereof. The lysate used is therefore formed in whole or in part of the intracellular biological constituents and of the constituents of cell walls and membranes of the microorganism under consideration.

More particularly, this lysate can be obtained following the technology described in U.S. Pat. No. 4,464,362, and in particular by ultrasound-induced disruption to release the cytoplasmic fractions, cell wall fragments and some products derived from the metabolism of these microorganisms.

The extract of interest can be obtained from any material derived from a whole fungus, or any part of said fungus e.g. the cap, stipe, mycelium, and mixtures thereof.

Preferably, the extract of interest is obtained from any fresh material and hence not having undergone a drying, freeze-drying or freezing step.

The production (extraction) of an extract of interest lies within the general knowledge of those skilled in the art for whom several techniques are available such as filtration, pressing, decoction, enfleurage, infusion, maceration, steam distillation (or hydrodistillation), solvent extraction, Soxhlet extraction, batch extraction, ultrasound-assisted extraction, microwave-assisted extraction, accelerated solvent extraction, supercritical fluid extraction, extraction based on the principle of sonoporation (e.g. Eco-Sound® technology by Croda), medium-pressure solid-liquid extraction—MSLE based on the following principle: the solvent, fed via a pump, passes through a column under a certain pressure (˜10 bar) that is filled with material to be extracted (fixed bed column), ultrasound disruption, and combinations thereof.

Preferably, the extraction technique is a technique heedful of the environment, in particular one that is energy-saving and/or when necessary has recourse to «clean» solvents e.g. glycerine, water, ethanol, isopropanol, propanediol, and mixtures thereof.

Evidently, it is within the reach of skilled persons to adjust/select the appropriate extraction method depending on the plant, microorganism and/or fungus under consideration and/or in the light of the desired extract of interest. These adjustments and choices lie within the general knowledge of skilled persons.

### Third Section

Optionally, a unit of the invention also comprises a third section dedicated to the manufacture of a composition particularly a cosmetic composition comprising at least said extract of interest and a physiologically acceptable medium.

In a first embodiment, the first, second and third sections are located within one same building (or enclosure).

In another embodiment, the third section is separate from the first section and second section, the third section nevertheless being adjacent to at least the second section.

In the meaning of the present invention, a «composition» can also be termed an «extemporaneous composition» when it is produced within a very short interval of time after production of the extract of interest.

For example, an extemporaneous composition of the invention, particularly a cosmetic composition, can be manufactured in less than 24 hours, preferably less than 12 hours, in particular in less than 5 hours, better still in less than 3 hours even in less than one hour after production of the extract of interest, even after the harvesting of the plant, microorganism and/or fungus used to produce the extract of interest. This embodiment is advantageous in that it can reduce and even prevent any phenomenon of deterioration of the extract of interest before it is incorporated in a composition particularly a cosmetic composition.

A composition of the invention is advantageous in that it comprises at least one fresh active ingredient, namely the extract of interest and a physiologically acceptable medium, and is advantageously free of pesticide.

Preferably, a composition of the invention can be a cosmetic, pharmaceutical, nutraceutical or food composition.

Preferably, a composition of the invention is a cosmetic composition, dedicated in particular to the care and/or enhancing of keratin material and the skin in particular.

The manufacture of a composition of the invention, and in particular the step to add (or incorporate) the extract of interest, lies within the general knowledge of persons skilled in the art.

In general, a composition of the invention also comprises at least one physiologically acceptable medium.

In the invention, and unless otherwise stated, by “physiologically acceptable medium” it is meant an appropriate medium for the application under consideration, in particular for cosmetic applications, and especially suitable for application of a composition of the invention onto keratin material in particular the skin and/or hair and more particularly the skin.

The physiologically acceptable medium is generally adapted to the type of medium onto which the composition is to be applied and to the type of packaging of the composition.

In one embodiment, the physiologically acceptable medium is an aqueous phase, preferably in gel form.

In another embodiment, the physiologically acceptable medium is an oil phase, preferably in gel form.

A composition of the invention can be in any form. For example a composition of the invention can be a cream, emulsion, (direct, inverse or multiple), lotion, serum, gel and oil for the skin (hands, face, feet, etc.), a foundation (liquid, paste) bath and shower preparation (salts, foams, oils, gels, etc.), hair care product (hair dyes and bleaches), cleaning product (lotions, powders, shampoos), hair conditioning products (lotions, creams, oils), hair styling product (lotions, lacquers, brilliantine), shaving product (soaps, foams, lotions, etc.), lip application product, sun product, self-tanning product, skin lightening product, anti-wrinkle product. In particular, a cosmetic composition of the invention can be an anti-ageing serum, youth serum, hydrating serum or scented water.

A food composition of the invention can be a yoghurt for example.

Preferably, a composition of the invention is an emulsion, preferably macroscopic i.e. in which the dispersed phase is in the form of droplets visible to the naked eye and having a diameter for example of between 100 μm and 3000 μm, preferably between 250 μm and 1500 μm, kinetically stable and optionally monodisperse.

An emulsion can be a direct emulsion (i.e. oil-in-water), inverse (i.e. water-in-oil) or multiple, in particular double (i.e. water-in-oil-in-water, oil-in-water-in-oil or oil-in-oil-in-water).

The monodisperse nature, and a measuring method are defined in FR3041511.

Advantageously, an extract of interest in used in a composition of the invention in encapsulated form.

In particular a composition of the invention, particularly one in which the extract of interest is used in encapsulated form, is manufactured following the method such as described in WO2010063937, WO2012120043 or WO2018167309.

Preferably, a composition of the invention comprises from 0.00001 weight % to 50 weight %, preferably from 0.0001 weight % to 10 weight %, and better still from 0.01 weight % to 5 weight % of extract(s) of interest relative to the total weight of said composition.

### Additional Compound(s)

A composition of the invention can also comprise at least one additional compound differing from the above-mentioned extract of interest and physiologically acceptable medium.

A composition of the invention may further comprise at least powders; glitter; colouring agents selected in particular from among colouring agents which may or may not be water-soluble or liposoluble, organic or inorganic, materials having an optical effect, liquid crystals, and mixtures thereof; particulate agents insoluble in the fatty phase; preserving agents; humectants; stabilizers; chelating agents; emollients; modifying agents selected from among texturizing, viscosifying agents, (e.g. aqueous phase gelling/texturizing agents differing from the above-mentioned base), pH modifiers, osmotic strength modifiers and/or refractive index modifiers etc . . . or any usual cosmetic additive; and mixtures thereof.

For example, the colouring agent is selected from among pigments, pearlescent agents (glitter), optical effect materials in particular liquid crystals and mixtures thereof, preferably pigments and/or pearlescent materials.

A composition of the invention may also comprise at least one biological/cosmetic active agent differing from the extract of interest. The active agent is advantageously a cosmetic, dermo-pharmaceutical, pharmaceutical or food agent.

A biological/cosmetic active agent can be selected from among hydrating agents, healing agents, depigmenting agents, UV filters, peeling agents, antioxidant agents, active agents stimulating synthesis of dermal and/or epidermal macromolecules, dermo-relaxant agents, anti-perspiration agents, soothing agents, anti-wrinkle agents, anti-irritation agents, tensor/smoothing agents, anti-cellulite agents, firming agents, sculpting agents, softening agents, emollient agents, slimming agents, protective/antiradical agents, draining agents, anti-inflammatory agents, lightening agents, self-tanning agents, exfoliating agents, cell renewal or skin microcirculation stimulating agents, absorbing agents, anti-dandruff agents and/or anti-aging agents, and mixtures thereof.

If the active agent is a pharmaceutical agent, it is advantageously selected from among anticoagulants, anti-thrombogenic agents, anti-mitotic agents, anti-proliferation, anti-adhesion, anti-migration agents, cell adhesion promoters, growth factors, antiparasitic molecules, anti-inflammatories, pro-angiogenic agents, angiogenesis inhibitors, vitamins, hormones, proteins, antifungals, antimicrobial molecules, antiseptics, antibiotics, and mixtures thereof.

Food agents are advantageously vegetable or fruit purees such as mango puree, pear puree, coconut puree, cream of onion, leek and carrot, or other preparations in which several fruits or vegetables can be mixed. As a variant, they can be an oil such as a food oil e.g. olive, soybean, grapeseed, sunflower seed oil or any other oil extracted from vegetables, and food active substances such as probiotics, yeasts, vitamins, minerals, oleo-active substances, and mixtures thereof.

A composition of the invention may also comprise excipients, such as thickeners or rheology modifiers. For example, these thickeners are polymers, cross-polymers, microgels, gums or proteins including polysaccharides, celluloses, polyosides, silicone-containing polymers and copolymers, colloidal particles (silica, clays, latex . . . ), and mixtures thereof.

The additional compound(s) in particular the active agent(s) can be hydrophilic or lipophilic.

Evidently those skilled in the art will seek to choose any additional compound(s) and/or the quantity thereof so that the advantageous properties of a composition of the invention, in particular the extract of interest, are not or not substantially changed through the envisaged addition. In particular, the type and/or quantity of the additional compound(s) will depend on the aqueous or fatty nature of the phase under consideration. These adjustments lie within the competence of skilled persons.

### Fourth Section

Optionally, a unit of the invention may further comprise a fourth section dedicated to the packaging in a container (also called primary packaging) of the composition particularly a cosmetic composition manufactured in the third section, and even dedicated to the packaging of said container in secondary packaging.

In a first embodiment, the first, second, third and fourth sections are located in the same building (or enclosure).

In another embodiment, the fourth section is separate from at least one section selected from among the first section, second section and third section, the fourth section at least being adjacent to the third section however.

The packaging of a composition of the invention can be carried out using any technique known to skilled persons. Said container can be in different forms e.g. a small dish, jar, tube, small or large bottle, optionally provided with an aspiration and dispensing device.

### Fifth Section

Optionally, a unit of the invention may also comprise a fifth section dedicated to the marketing of the extract of interest produced in the second section, even of the composition particularly a cosmetic composition manufactured in the third section or packaged in a container (primary packaging) or even packaged in secondary packaging in the fourth section.

A unit of the invention is advantageous since it concentrates together all the steps needed for the manufacture of an end product, in particular a cosmetic end product, supplied directly to the final consumer whilst paying heed in particularly satisfactory manner to the criteria of ecology and sustainable development.

A unit of the invention allows a significant reduction in the need for machine-tools as required within a conventional supply chain, and in energy and fossil carbon needs as well as in qualified staff, water resources, CO2 emissions and hence in production costs. This reduction applies to the cultivation of the plant, microorganism and/or fungus and to the production of the extract of interest derived from said plant, said microorganism and/or said fungus, but can also apply to the manufacture of an end product particularly a cosmetic product comprising said extract of interest and to the packaging and marketing thereof.

These advantages permit urban and local use of a unit of the invention, which allows «short supply» chains and thereby further limits the recourse to transport and storage chains that are costly in vehicles, machines and fuel.

For the production of an extract of interest, even of a composition particularly a cosmetic composition, that is easily and directly accessible to the final consumer, a unit of the invention therefore meets ecological and sustainable development criteria in particularly satisfactory manner whilst remaining coherent at industrial level and economically sound.

### EXAMPLES

This example illustrates a unit for the manufacture of two cosmetic compositions A and B each comprising an extract of interest derived from buckwheat.

This unit comprises 5 sections distributed as follows:
a first section dedicated to the cultivation of buckwheat up until the microgreen stage (i.e. 10 to 14 days starting from seeding up until harvesting) using a hydroponic system and under controlled conditions;
a second section adjacent to the first section and dedicated to the extraction based on the principle of sonoporation (i.e. Eco-Sound® technology) of buckwheat microgreens harvested in the first section, using a water solvent (100%) or mixture of water/glycerine type (50/50) to obtain an extract of interest;
a third section adjacent to the second section dedicated to the manufacture of the cosmetic compositions A and B comprising the extract of interest in the form of a direct emulsion, said emulsion comprising a continuous aqueous phase and dispersed oil phase. These two emulsions A and B were obtained with a microfluidic method such as described in WO2012120043 and comprised a dispersed phase in the form of macroscopic droplets (1000 microns) and monodisperse phase;
a fourth section adjacent to the third section and dedicated to the packaging of said cosmetic compositions in primary packaging and then secondary packaging; and
a fifth section adjacent to the fourth section and dedicated to the marketing of the compositions obtained in the fourth section;

the first section, second section, third section, fourth section and fifth section all being located in one and the same unit in the form of a shipping container modified and adapted to house said sections.

The container was also provided with photovoltaic panels to provide electrical power requirements in particular to provide light via LEDs and for the functioning of air conditioning and the air purification/treatment device, and comprised a station to capture and treat aquifer water to ensure water needs in particular for cultivation of the buckwheat, extraction of the extract of interest, manufacture of the cosmetic composition and cleaning of the different sections within the unit.

The cosmetic compositions A and B are described in Table 1 below.

TABLE 1

| | | | A | B |
|---|---|---|---|---|
| | Name | INCI | % w/w | % w/w |
| Dispersed | Lanol 99 | Isononyl | Q.S.* | 0 |
| fatty phase | | Isononanoate | | |
| (IF) | DUB 810 C | Coco- | 0 | Q.S.* |
| | | Caprylate/Caprate | | |
| | Rheopearl KL2 | Dextrin Palmitate | 5 | 0 |
| | Estogel M | CASTOR OIL/IPDI | 0 | 10 |
| | | COPOLYMER & | | |
| | | CAPRYLIC/ | | |
| | | CAPRIC | | |
| | | TRIGLYCERIDE | | |
| | CAS3131 | Amodimethicone | 0.2 | 0 |
| | Total | | 100 | 100 |
| Continuous | Reverse osmosis | Aqua | Q.S.* | Q.S.* |
| aqueous | (RO) water | | | |
| phase | Glycerol | Glycerin | 7.00 | 7.00 |
| (OF) | Zemea | Propanediol | 6.00 | 6.00 |
| | Microcare PTG | Pentyleneglycol | 2.35 | 2.35 |
| | Microcare PE | Phenoxyethanol | 0.95 | 0.95 |
| | Carbopol® Ultrez 10 | Carbomer | 0.24 | 0.24 |
| | polymer | | | |
| | Rhodicare T | Xanthan | 0.12 | 0.12 |
| | Extract of interest - | — | 3.5 | 3.5 |
| | buckwheat | | | |
| | Total | | 100 | 100 |
| Base | Reverse Osmosis | Aqua | 99.7 | 99.7 |
| (BF) | (RO) water | | | |
| | NaOH | Sodium Hydroxide | 0.3 | 0.3 |

* Q.S.: as much as is sufficient

### Preparation Protocol

### For OF

The carbomer is dispersed in the RO water and left under agitation for 2 hours using a radial flow impeller.
The glycerine, Propanediol (Zemea) and xanthan are added thereto. After these additions, the mixture is left under agitation for 10 min.
Phenoxyethanol (Microcare PE) and Pentyleneglycol (Microcare PTG) are added. The mixture is left under agitation for 5 min.
The extract of interest followed by sodium hydroxide are added under agitation.
At the last step, the mixture is mixed until homogenization is reached.

For the base (BF): The sodium hydroxide and water are mixed with a magnetic stir bar for 5 min.

### For IF

If used, the amodimethicone is added to the oil and mixed with a magnetic stir bar for 5 min.
The oil is heated (when applicable in the presence of amodimethicone) to 80° C. after which Rheopearl KL2 or Estogel M is added under magnetic stirring.
This mixture can then be placed over a hot water bath heated to 80° C. under magnetic stirring for 1 hour.

The IF solution heated to 80° C. was placed in a syringe connected to a heat source to maintain a hot solution. To reduce heat losses, the microfluidic device was positioned directly at the outlet of the syringe.

The microfluidic parameters for manufacture of the cosmetic composition are described in Table 2 below.

TABLE 2

| Phase | Nozzle flow rate (in mL/hr) |
|---|---|
| Continuous aqueous phase (OF) | 240 |
| Dispersed fatty phase (IF) | 20 |
| Base (BF) | 28.8 |

What is claimed is:

1. A unit for producing at least one extract of interest from at least one plant, at least one microorganism and/or at least one fungus, comprising at least two sections:
a first section dedicated to the cultivation, under controlled conditions, of at least one plant, at least one microorganism and/or at least one fungus; and
a second section dedicated to the production of the extract of interest derived from said plant and/or said microorganism and/or said fungus.

2. The unit according to claim 1, wherein cultivation of the plant is hydroponic or aquaponic cultivation.

3. The unit according to claim 1, wherein the plant is selected from among plants, in particular mosses, ferns, gymnosperms and angiosperms;
algae; and mixtures thereof.

4. The unit according to claim 1, wherein the plant is selected from among garlic, alfalfa, almond, amaranth, dill, arugula, oats, azukis, basil, Swiss chard, beetroot, leaf beet, wheat, broccoli, kaniwa, carrots, celery, chervil, chia, chicory, cabbage, scallions, chives, coriander, watercress, lady's smock, spelt, spinach, fennel, fenugreek, squash, sunflower, mung bean, kale, oriental wheat, lentils, red lentils, green lentils, flax, corn, sorghum, mizuna, mustard, turnip, onion, barley, parsley, leek, chick peas, peas, purslane, quinoa, radish, horseradish, rice, rocket, buckwheat, rye, sesame, soybean, tatsoi, trefoil, cannabis, and mixtures thereof.

5. The unit according to claim 1, wherein the microorganism is selected from among the genii Saccharomyces, Yarrowia, Kluyveromyces, Torulaspora, Schizosaccharomyces, Debaromyces, Candida, Pichia, Aspergillus, Penicillium, Bacteroides, Fusobacterium, Melissococcus, Propionibacterium, Enterococcus, Lactococcus, Staphylococcus, Peptostrepococcus, Bacillus, Pediococcus, Vitreoscilla, Micrococcus, Leuconostoc, Weissella, Aerococcus, Oenococcus, Bifidobacterium, Lactobacillus, and mixtures thereof.

6. The unit according to claim 1, wherein the fungus is selected from among Fungi, oomycetes, chytridiomycetes, mycetozoa and mixtures thereof, preferably from among the genii Agaricus, Lentinula, Pleurotus, Grifola, Hypsizygus, Auricularia, Flammulina, Volvariella, Agrocybe, Pholiota, Tricholoma, Tremella, and mixtures thereof.

7. The unit according to claim 1, wherein the plant extract is obtained from a sprouted seed, microgreen or young shoot, preferably a microgreen, and/or the microorganism extract is obtained from a microorganism in living, semi-active, inactivated or dead form.

8. The unit according to claim 1, wherein extraction is selected from among filtration, pressing, decoction, enfleurage, infusion, maceration, steam distillation, solvent extraction, Soxhlet extraction, batch extraction, ultrasound-assisted extraction, microwave-assisted extraction, accelerated solvent extraction, supercritical fluid extraction, extraction based on the principle of sonoporation, medium-pressure solid-liquid extraction, ultrasound disruption, and combinations thereof.

9. The unit according to claim 1, wherein the unit also comprises a third section dedicated to the manufacture of a composition, particularly a cosmetic composition, comprising the extract of interest and at least one physiologically acceptable medium.

10. The unit according to claim 9, wherein the composition is an emulsion, preferably macroscopic and optionally monodisperse.

11. The unit according to claim 9, wherein the unit also comprises a fourth section dedicated to the packaging of said composition, particularly a cosmetic composition, in a container.

12. The unit according to claim 1, wherein the unit also comprises a fifth section dedicated to the marketing of the extract of interest, even of the composition, particularly a cosmetic composition.

13. A method for producing an extract of interest, even a composition, particularly a cosmetic composition, comprising at least said extract of interest and a physiologically acceptable medium, comprising a step of providing the unit of claim 1.

14. (canceled)

15. A method for producing at least one extract of interest from at least one plant, at least one microorganism and/or at least one fungus, said method comprising at least the steps of:
(a) providing at least one unit according to claim 1;
(b) cultivating under controlled conditions at least one plant and/or at least one microorganism and/or at least one fungus;
(c) extracting the extract of interest from the plant and/or microorganism and/or fungus harvested at step (b);
(d) optionally, manufacturing a composition, particularly a cosmetic composition, comprising at least the extract of interest and a physiologically acceptable medium; and
(e) optionally, packaging said composition in a container, at least steps (b) to (c) being conducted within the unit.

16. The method according to claim 14, wherein steps (b) to (d), even (b) to (e), are conducted within the unit.

17. The method according to claim 15 said method between steps (b) and (c) also comprising at least one intermediate step (f) for freezing, dry-freezing, drying, grinding and/or centrifugation of the plant and/or microorganism and/or fungus.
